(19) United States
(12) Plant Patent Application Publication
(10) Pub. No.: US 2013/0347155 P1
(43) Pub. Date: Dec. 26, 2013

(54) Grapefruit tree named 'JR13'

(50) ;

(71) Applicant: Leon Esselen, Malelane (ZA)

(72) Inventor: Leon Esselen, Malelane (ZA)

(21) Appl. No.: 13/507,378

(22) Filed: Jun. 22, 2012

(51) Int. Cl. A01H 5/00

(52) U.S. Cl. PLT/201

(57) ABSTRACT

‘JR13’ is a new and distinct grapefruit tree notable for its attractive, seedless fruit.

### GENUS AND SPECIES

Citrus×paradisi

### VARIETY DENOMINATION

‘JR13’

### BACKGROUND AND SUMMARY OF THE VARIETY

The new grapefruit variety ‘JR13’ originated in Malelane, South Africa in 2005 as an induced mutation of ‘FE1’ grapefruit (not patented). Budwood was taken from the original selection and budded to ‘Carrizo’ citrange rootstock, and the new trees of ‘JR13’ were planted at Malelane. ‘JR13’ has been observed over successive generations and has been found to retain its distinguishing characteristics.

‘JR13’ is a new and distinct grapefruit tree notable primarily for its attractive, seedless fruit. Table 1 shows a comparison of ‘JR13’ to parent ‘FE1’ and ‘Marsh’ (not patented) grapefruit varieties.

TABLE 1
Comparison of ‘JR13’ to Known Varieties

| Characteristic | ‘JR13’ | ‘FE1’ | ‘Marsh’ |
|---|---|---|---|
| Seed quantity | Seedless | Some seeds | Some seeds |
| Maturity | Mid- to late-season | Mid-season | Early season |
| Fruit shape | Oval | Round | Round |

### BRIEF DESCRIPTION OF THE PHOTOGRAPHS

FIG. 1 shows whole and sectioned fruit of the ‘JR13’ grapefruit tree. Because lighting conditions can affect the colors shown in photographic illustrations, color characteristics of this new variety should be determined with reference to the observations described herein, rather than from the illustrations alone.

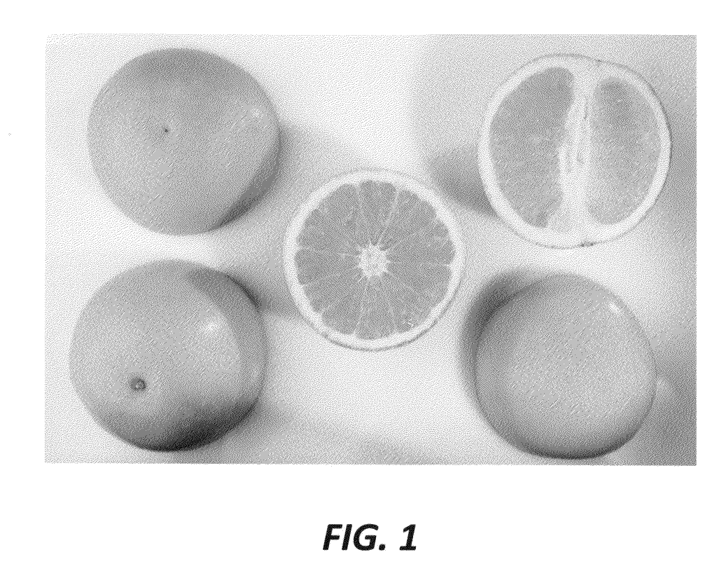

### DETAILED BOTANICAL DESCRIPTION

The following detailed botanical description is based on observations of trees and fruit of ‘JR13’, budded to ‘Carrizo’ citrange rootstock and planted in 2010. Observations were recorded and photographs taken during the 2011 growing season at Malelane, South Africa. It should be understood that the characteristics described will vary somewhat depending upon cultural practices and climatic conditions, and can vary with location and season. Quantified measurements are expressed as an average of measurements taken from a number of individual plants of the new variety. The measurements of any individual plant, or any group of plants, of the new variety may vary from the stated average.
Tree:
   Vigor.—Moderate.
   Density of canopy.—Dense.
   Habit.—Compact growth habit.
   Height.—1.60 m.
   Trunk diameter (at 30 cm above the graft).—18 cm.
   Bark texture.—Smooth.
   Bark color.—White with green stripes.
   Tendency toward alternate bearing.—No.
Branch (fruiting branches located at around 1 m above the graft union):
   Length.—25 cm.
   Diameter.—2.8 cm.
   Crotch angle.—25-30°.
   Bark color.—Green.
   Bark texture.—Smooth.
   Thorns present? .—Yes.
   Thorn length.—10 mm.
   Current year shoot length.—30 cm.
   Current year shoot color.—Green to light green.
Leaves:
   Length.—12 cm.
   Width.—7 cm.
   Length:width ratio.—1:0.6.
   Blade margin.—Smooth.
   Leaf shape.—Oval.
   Apex shape.—Acute.
   Color of upper surface.—Green.
   Color of lower surface.—Light green, yellow.
   Leaf texture.—Smooth.
   Petiole length.—21.3 mm.
   Petiole diameter.—17.7 mm.
   Petiole color.—Light green.
   Wings present? .— Yes.
   Wing width.—9 mm.
Fruit:
   Quantity per cluster.—1 to 7.
   Axial diameter.—77.2 mm.
   Apical diameter.—78.8 mm.
   Weight.—259 g.
   General shape in profile.—Round.
   Navel.—Absent.
   Rind color.—Yellow to deep yellow.
   Oil glands per cm².—19.
   Oil gland diameter.—1.3 mm.
   Rind thickness.—5.5 mm.
   Ease of peeling.—Difficult.
   Rind texture.—Smooth.
   Albedo thickness.—3.1 mm.
   Albedo color.—White.
   Quantity of fruit segments per fruit.—12.
   Toughness of segment membrane.—Moderate.
   Juice sac length.—19.8 mm.
   Juice sac shape.—Elongated.
   Juice sac length to width ratio.—1:6.6.
   Juice sac color.—White, tan.
   Juice soluble solids (*Brix).—13.1.
   Relative harvest maturity.—Mid- to late-season.
   Harvest window.—4 weeks.
   Seeds present.—None.
   Stem length.—3-4 cm.
   Stem diameter.—3.3 mm.
   Stem color.—Light green.
   Market use.—Fresh, juice and segments.
   Keeping quality.—Good.
   Shipping quality.—Good.

1. A new and distinct variety of grapefruit tree, substantially as illustrated and described herein.
